(19) United States
(12) Patent Application Publication
(10) Pub. No.: US 2025/0186659 A1
(43) Pub. Date: Jun. 12, 2025

(54) FABRICATION METHOD OF DIRECTIONAL TISSUE WITH ACOUSTIC PATTERNING OF PORE- FORMING PARTICLES

(71) Applicant: UIF (University Industry Foundation), Yonsei University, Seoul (KR)

(72) Inventors: Hyung Suk LEE, Seoul (KR); Yu Nam LEE, Seoul (KR); Byung Jun KANG, Seoul (KR); Chan Ryeol RHYOU, Seoul (KR)

(73) Assignee: UIF (University Industry Foundation), Yonsei University, Seoul (KR)

(21) Appl. No.: 18/923,379

(22) Filed: Oct. 22, 2024

(30) Foreign Application Priority Data
Oct. 23, 2023 (KR) 10-2023-0142452

(51) Int. Cl. A61L 27/56; A61L 27/36; A61L 27/38; A61L 27/52; B29C 67/20; B29K 105/00; B29L 31/00

(52) U.S. Cl. CPC A61L 27/56; A61L 27/3691; A61L 27/3895; A61L 27/52; B29C 67/20; A61L 2400/08; A61L 2430/34; B29K 2105/0061; B29L 2031/7532

(57) ABSTRACT

Disclosed is a method of applying a three-dimensional acoustic wave to cells and pore-forming particles similar in size to cells contained in a hydrogel to perform the micro-sized patterning thereof, and then melting the particles to form pores in which the tissue is rapidly cultured and is regenerated.

### BACKGROUND

### Field

The present disclosure relates to a method for fabrication of a directional tissue or artificial blood vessel using acoustic patterning of pore-forming particles and to a directional tissue or artificial blood vessel fabricated using the method.

### Description of Related Art

Various tissues in the body, such as muscles, nerves, and blood vessels, have anisotropic structures. In order to increase the efficiency of movement and material transfer thereof, they have a linear pattern. Thus, copying this linear pattern has become a major task in tissue engineering. In particular, there is a growing need for fabricating an alternative test method model that maintains the structure of the tissue and expresses its function in vitro. However, existing technologies such as bio-3D printing, molding, and laser ablation have limitations in copying the integrated tissue structure. Existing technologies have low transplantable cell concentrations and physical resolution exceeding 100 micrometers (μm), thereby making it impossible to copy the capillary structures arranged within 100 micrometers (μm) like those in living organisms.

On the other hand, acoustic wave patterning technology may efficiently create highly functional tissues by patterning cells at spacings of tens to hundreds of micrometers (μm). In particular, surface acoustic waves among acoustic waves not only precisely pattern particles, but are also cell-friendly in a non-invasive manner. When the surface acoustic waves are used to generate standing waves in a solution containing high-concentration cells, the cells move to nodes arranged at spacings of 100 micrometers. Therefore, this scheme is suitable for copying micro blood vessels, muscles, etc. Furthermore, as a cell concentration increases locally, cell-to-cell interaction increases, thereby increasing the expression of biological markers necessary for tissue regeneration. However, it is difficult to achieve efficient tissue regeneration using simple patterns alone. This is because the topological cue of the extracellular matrix is insufficient. In order for cells to form tissues, they must secrete matrix metalloproteinases to dissolve the extracellular matrix, and migrate and proliferate. However, it takes a long time to secrete growth factors and remodel the extracellular matrix. Therefore, when pores are pre-formed so as to be arranged in the direction in which the cells grow, tissues may be fabricated quickly and efficiently.

According to the present disclosure, cells and pore-forming particles may be aligned, that is, patterned in one direction using acoustic waves to rapidly fabricate tissues. Pores formed by melting pore-forming particles adjacent to the cells provide an environment suitable for cell migration and division. They may move into the pores and may proliferate, and connect with adjacent cells, and quickly form the tissues through cell-to-cell interaction. Furthermore, the pores facilitate the transfer of substances within the hydrogel, helping the growth of thick tissues. The pores induce the rapid formation of hollow cylindrical structures (lumens) like pipes, such as blood vessels and lymph. Furthermore, the thickness of the tissue unit may be controlled by controlling the concentration of pore-forming particles.

Rapid fabrication of tissues using primary cells enables in vitro drug screening. Furthermore, a patient-specific drug response test platform may be rapidly mass-produced. Blood vessels, pore-forming particles, and patient-derived organoids may be patterned together to implement capillaries and the drug tests may be performed.

Since substance exchange is easily performed through the pores formed in the hydrogel, thick tissue regeneration may be achieved. When the patient's muscle, nerve, and vascular cells are patterned together with pore-forming particles and transplanted to the area where damage has occurred, rapid treatment may be achieved. Mass production of artificial meat may be achieved by fabricating the thick muscle tissue.

With existing technologies, it takes a long time to fabricate a 3D vascular system. In the case of sponge scaffolds and electrospinning scaffolds, cells cannot be added during the scaffold fabrication process. Therefore, it takes a long time for cells to move and proliferate and then grow into blood vessels. When adding and culturing cells in hydrogel, the size of the gel should be limited due to the limitations in the diffusion of nutrients, oxygen, and carbon dioxide. Furthermore, the tissue morphology within the hydrogel grows irregularly, making it difficult to quantify changes in blood vessels through experimental manipulation.

There are limitations in patterning cells using electric or magnetic methods. In order to perform cell electropatterning, electrodes must be connected to the sample, which may cause physical deformation of the sample or changes in the electrical properties inside the cell. Magnetic patterning technology may be used to pattern cells by exposing them to a magnetic field. In order to react the cells with the magnetic field, magnetic nanoparticles must be attached to the cell surface. However, there is a problem that the coating material of the particles is toxic, and causes toxicity depending on the size and concentration of the particles. Therefore, particle removal is essential. Thus, the tissue damage may occur during the removal step.

### SUMMARY

The present disclosure is intended to solve the problems of the above-mentioned prior art, and thus a purpose of the present disclosure is to provide a method for fabricating a porous hydrogel including cells and pore-forming particles, and an artificial tissue fabricating device using acoustic patterning of pore-forming particles.

In order to achieve the purpose, the present disclosure provides a method for fabricating a hydrogel structure in which micro-sized pores may be formed so as to be arranged in a direction of tissue growth, the method comprising: a first step of mixing liquid hydrogel with cells and pore-forming particles; a second step of applying an acoustic wave in one or more directions to the liquid hydrogel with which the cells and the pore-forming particles have been mixed, thereby aligning the cells and the pore-forming particles with each other; a third step of curing the liquid hydrogel in which the cells and the pore-forming particles may be aligned with each other, thereby forming a hydrogel structure; and a fourth step of removing the pore-forming particles from the hydrogel structure to form pores inside the hydrogel structure.

In one embodiment of the present disclosure, the second step may include applying an acoustic wave to the liquid hydrogel to pattern the cells and the pore-forming particles.

In one embodiment of the present disclosure, the second step may include applying the acoustic wave to the liquid hydrogel to pattern the cells and the pore-forming particles in one direction.

In one embodiment of the present disclosure, the acoustic wave may be a standing wave.

In one embodiment of the present disclosure, the hydrogel may be cured before removing the pore-forming particles in the fourth step.

In one embodiment of the present disclosure, in the fourth step, the pore-forming particles may be dissolved and removed using at least one of a solvent, heat, or light.

In one embodiment of the present disclosure, in the fourth step, the pore-forming particles may be dissolved and removed using the heat.

In one embodiment of the present disclosure, the method may further comprise a fifth step of growing cells into the pores obtained by removing the pore-forming particles, thereby forming a tissue.

Further, the present disclosure provides a porous hydrogel for tissue growth, wherein the porous hydrogel may be fabricated using the method for fabricating the porous hydrogel as described above, wherein the porous hydrogel for tissue growth comprises: a hydrogel body; pores formed within the hydrogel body; and a cell group positioned adjacent to the pores.

Further, the present disclosure provides an artificial tissue fabricating device using acoustic patterning of pore-forming particles, the artificial tissue fabricating device comprising: a chamber capable of receiving therein hydrogel, cells, and pore-forming particles; and an acoustic patterning unit capable of applying an acoustic wave to the chamber.

In one embodiment of the present disclosure, the acoustic patterning unit may include a piezoelectric element substrate, and an IDT (Interdigital transducer) electrode disposed on the piezoelectric element substrate.

In one embodiment of the present disclosure, the acoustic patterning unit may be configured such that an alternating current may be applied to the IDT electrode to generate the acoustic wave.

In one embodiment of the present disclosure, the acoustic wave may be a surface acoustic wave oscillating from the piezoelectric element substrate.

In one embodiment of the present disclosure, the acoustic wave may be applied from the acoustic patterning unit to the chamber such that the cells and the pore-forming particles may be patterned in one direction within the hydrogel.

In one embodiment of the present disclosure, the acoustic patterning unit may be configured to apply a standing wave to the chamber.

In one embodiment of the present disclosure, the artificial tissue fabricating device may further comprise an acoustic coupling medium further disposed between the acoustic patterning unit and the chamber.

Using the method for fabricating the porous hydrogel and the artificial tissue fabricating device using acoustic patterning of pore-forming particles of the present disclosure, a three-dimensional acoustic wave is applied to the cells and pore-forming particles similar in size to cells in the hydrogel to perform micro-sized patterning thereof, and then the particles are melted to form the pores while simultaneously rapidly culturing and regenerating tissue.

### BRIEF DESCRIPTION OF DRAWINGS

FIG. 1A shows movement of a cell toward a pore when the pore is formed adjacent to the cell.

FIG. 1B shows tissue formation when pores are absent, and tissue formation when the pores are arranged in a straight line and are adjacent to cells.

FIG. 2 shows the pore-forming particles and the state after the pore-forming particles are removed.

FIG. 3A to FIG. 3F show various types of patterned tissue units.

FIG. 4 is a schematic diagram of an artificial tissue fabricating device of the present disclosure.

FIG. 5 is an illustration of artificial vascular tissue regeneration.

FIG. 6 is a flow chart of an artificial tissue fabricating process.

FIG. 7 is a schematic diagram of the operation of the device during the artificial tissue fabricating process.

FIG. 8 is a schematic diagram of structure decomposition and tissue extraction after tissue fabrication.

FIG. 9 is an image of the artificial vascular tissue fabricated according to the present disclosure, which is observed using a confocal microscope after staining actin as the cell structure with phalloidin.

FIG. 10 is an image of the aligned microvascular structure after culturing the artificial vascular tissue fabricated according to the present disclosure for 2 days.

### DETAILED DESCRIPTIONS

Hereinafter, embodiments of the present disclosure will be described in detail with reference to the attached drawings. The present disclosure may be modified in various ways and may take various forms, and thus, specific embodiments are illustrated in the drawings and described in detail herein. However, this is not intended to limit the present disclosure to a specific disclosure form. Rather, it should be understood that the present disclosure includes all modifications, equivalents, or substitutes included in the spirit and technical scope of the present disclosure. Similar reference numerals are used for similar components while describing the drawings.

The terms used in this application are used only to describe specific embodiments and are not intended to limit the present disclosure. As used herein, the singular constitutes “a” and “an” are intended to include the plural constitutes as well, unless the context clearly indicates otherwise. It will be further understood that the terms “comprise”, “comprising”, “include”, and “including” when used in the present disclosure, specify the presence of the stated features, integers, operations, elements, and/or components, but do not preclude the presence or addition of one or more other features, integers, operations, elements, components, and/or portions thereof.

A method for fabricating a porous hydrogel structure according to the present disclosure may include a first step of mixing liquid hydrogel with cells and pore-forming particles; a second step of applying an acoustic wave in one or more directions to the liquid hydrogel with which the cells and the pore-forming particles have been mixed, thereby aligning the cells and the pore-forming particles with each other; a third step of curing the liquid hydrogel in which the cells and the pore-forming particles may be aligned with each other, thereby forming a hydrogel structure; and a fourth step of removing the pore-forming particles from the hydrogel structure to form pores inside the hydrogel structure.

In the context of the present disclosure, ‘pore-forming particles’ may mean particles including any material that may be dispersed in the continuous phase and then removed by a subsequent process to form pores in the continuous phase. The size of the cell or tissue unit (spheroid, organoid, etc.) is in a range of 5 to 100 micrometers (μm), and the size of each of the pore-forming particles may be in a range of 0.5 to 2 times thereof. However, the size thereof is not limited as long as the pore-forming particle can form the pore that the cell or tissue unit may detect and protrude into or enter. Accordingly, the frequency of the applied acoustic wave may be in the range of 1 MHz to 100 MHz to form a tissue spacing. In one example, the tissue spacing was set to 140 μm using 14 MHz as the frequency of the acoustic wave. In this regard, an error of the spacing may occur depending on the device and the oscillation frequency of the acoustic wave. However, theoretically, tissues may be formed at a spacing of 140 μm.

In one embodiment of the present disclosure, the second step may include applying an acoustic wave to the liquid hydrogel to pattern the cells and the pore-forming particles.

In the context of the present disclosure, the patterning may mean an act of causing a target concentration gradient of a target substance to occur in at least a partial area. Applying the acoustic wave to the liquid hydrogel may allow the cells and the pore-forming particles to be present in a higher concentration in the partial area of the hydrogel structure than that in the other area of the hydrogel structure. As long as the patterning causes the target concentration gradient thereof, the type of the acoustic wave, the direction of application of the acoustic wave, the number of directions of application of the acoustic wave, etc. are not particularly limited.

In one embodiment of the present disclosure, the second step may include applying the acoustic wave to the liquid hydrogel to pattern the cells and the pore-forming particles in one direction.

In the context of the present disclosure, “patterning the cells and the pore-forming particles in one direction” means arranging the cells and the pore-forming particles to form a channel-like structure in at least a specific direction. This does not mean that the channel-like structure oriented in the specific direction is single, or that there is no target patterned material between the channel-like structures. Therefore, when the cells and the pore-forming particles are patterned in one direction, one or more structures in which cells and pore-forming particles having a significantly high concentration are arranged in at least one direction may be formed, and the cells and pore-forming particles having a significant concentration or higher may exist between the structures. This means a continuous linear shape. That is, not a straight line but a shape of a curve or a grid pattern may be formed. For example, the method of the present disclosure may fabricate a circular or grid pattern by controlling the chamber or a shape of an acoustic wave.

In one embodiment of the present disclosure, the acoustic wave may be a surface acoustic wave or a standing wave.

In the context of the present disclosure, the standing wave may mean a wave form in which a node is fixed within a significantly narrow range. Applying the standing wave to the hydrogel to pattern the cells and the pore-forming particles may allow the cells and the pore-forming particles to be stably patterned in one direction.

In one embodiment of the present disclosure, the hydrogel may be cured before removing the pore-forming particles in the fourth step.

Curing the hydrogel may allow the cells and the pore-forming particles to be fixed without diffusion within a significantly short period of time in a state in which the cells and the pore-forming particles have been patterned via the application of the acoustic wave. Thus, pores in which the cells may grow in the patterned state may be provided when the pore-forming particles are removed in a subsequent process.

In one embodiment of the present disclosure, in the fourth step, the pore-forming particles may be dissolved and removed using at least one of a solvent, heat, or light.

In one embodiment of the present disclosure, in the fourth step, the pore-forming particles may be dissolved and removed using the heat. This is merely an example, and the scheme of removing the pore-forming particles is not particularly limited.

In one embodiment of the present disclosure, the method may further comprise a fifth step of growing cells into the pores obtained by removing the pore-forming particles, thereby forming a tissue. The cells may grow into the hydrogel structure, and may grow into the empty space (the pore) while being supported by the hydrogel structure. In this regard, it is easier for the cell to grow into the empty space while being supported by the hydrogel structure, and a growth rate is high when the cell grows into the empty space while being supported by the hydrogel structure. Thus, the cell may grow into the pores obtained by removing the pore-forming particles, thereby forming a structure patterned in one direction.

The type of the cells is not particularly limited, and any cell that is advantageous in forming a tissue having a structure patterned in one direction may be preferable. Non-limiting examples of the cells may include vascular cells, muscle cells, nerve cells, etc.

The concentration at which the pore-forming particles are contained in the hydrogel is not particularly limited. However, when the pore-forming particles are contained at an excessively low concentration, the growth of the cells may proceed excessively slowly due to insufficient pores. In this case, it may take an excessively long time for the cells to grow such that they have sufficient self-supporting capacity after the hydrogel is removed. Furthermore, when the pore-forming particles are contained at an excessively high concentration, the hydrogel structure may not have sufficient self-supporting capacity after the pore-forming particles are removed, and/or a structure usefully patterned in one direction may not be formed even when the cells grow. Therefore, the pore-forming particles of an appropriate concentration depending on the type of the pore-forming particles and a target cell structure should be contained in the hydrogel structure. In a non-limiting example, the content of the pore-forming particles may be in a range of 1/20 to 2/3 of a total volume of the hydrogel structure. Depending on the size or the type of the pore-forming particles, approximately 10⁷ mL⁻¹ of the pore-forming particles may be contained in the hydrogel.

Hereinafter, a method for fabricating a porous hydrogel according to the present disclosure is described in detail.

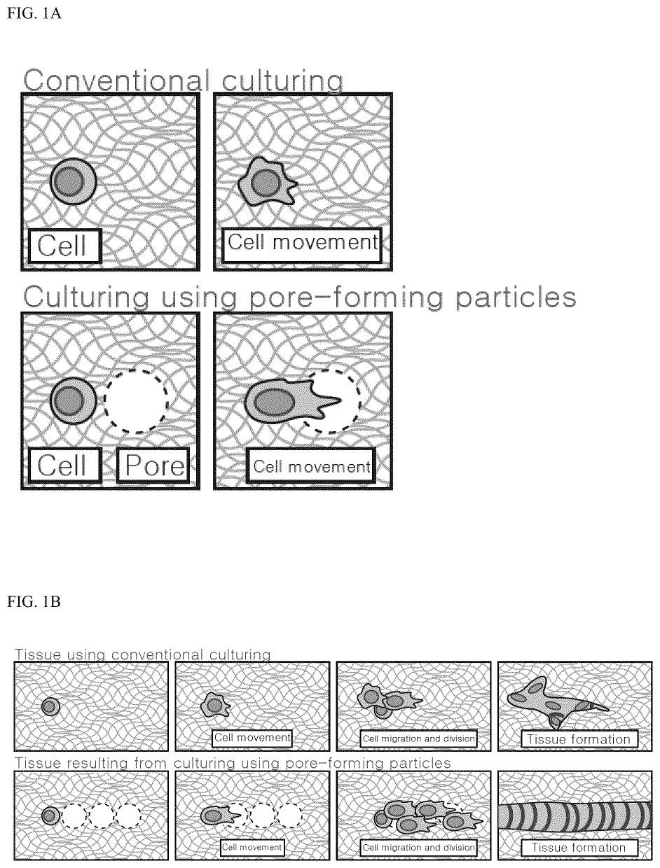

FIG. 1A shows movement of a cell toward a pore when the pore is formed adjacent to the cell.

FIG. 1B shows tissue formation when pores are absent, and tissue formation when the pores are arranged in a straight line and are adjacent to cells.
(1) Orientation of cells toward pore and tissue fabricating period

Referring to FIG. 1A and FIG. 1B, cells in a 3D hydrogel move and grow while penetrating the gel. At this time, they move to a point where gel deformation is relatively easy. Therefore, the time for the cells to modify the gel is greatly reduced, thereby making it easy for the cells to grow into tissues. While the conventional technology takes 4 to 14 days to fabricate the blood vessel, the method of the present disclosure may fabricate the blood vessel in just 2 days using the orientation of cells toward the pore. Furthermore, the fabricating period of a tissue other than the blood vessel may be reduced by more than half using the method of the present disclosure.

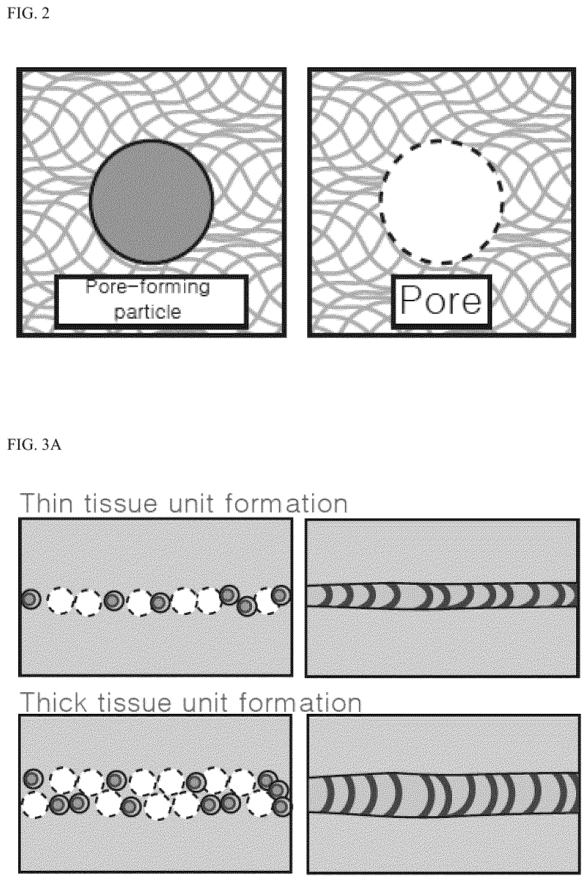

FIG. 2 shows the pore-forming particles and the state after the pore-forming particles are removed.
(2) Pore formation principle

Referring to FIG. 2, the principle of the pore formation uses gelatin (or PNIPAAm, etc.) as a temperature-variable gel to form the pores in the hydrogel. The gelatin is converted to a gel at low temperatures and becomes liquid when the temperature thereof reaches a body temperature. The liquid gelatin is fabricated into a bead shape using a water-in-oil emulsion, followed by washing several times with saline solution. Hydrogel may be cured before removing the pore-forming particles. When these beads are placed into the hydrogel and the hydrogel is converted to the gel and then the temperature thereof is raised to the body temperature such that the gelatin becomes liquid and then is eluted into the hydrogel matrix, leaving behind the pores. In addition to temperature variability, enzymatic treatment (agarose, gelatin, etc.) may be applied or a pH change (collagen, gelatin, albumin, etc.) may be induced, such that the pore-forming particles may be fabricated. The pore-forming particles may be dissolved and removed using at least one selected from the group containing solvent, heat, or light. Therefore, the pore-forming particles may be made of water-soluble gels, photosensitive gels, or photodegradable gels.

In other words, the pores may be formed by utilizing a difference between the decomposition rates of the hydrogel and the pore-forming particles. That is, the principle that the pore-forming particles decompose faster than hydrogel to form the pores due to the matrix remodeling mechanism (extracellular remodeling) of the cells or physical/chemical factors may be utilized.
(3) Hydrogel cell pattern

The patterning may be performed by applying the acoustic wave to the hydrogel to align the cells and the pore-forming particles with each other in the hydrogel in one direction. A standing surface acoustic wave is used to align the cells and the pore-forming particles with each other in the hydrogel. The surface acoustic wave is applied thereto using a piezoelectric substrate or an acoustic wave transmitting material. The particles and the cells are aligned with each other in one direction via the application of the acoustic wave, and the pore-defining beads are melted to form the pores. In this regard, the spacing of the pattern is 140 micrometers in a left-right direction and 50 micrometers in a vertical direction. This size is suitable for mimicking a ratio of the biological blood vessel. After removing the pore-forming particles, the cells may be grown into the pores obtained by removing the pore-forming particles, thereby forming the tissue.

Further, a porous hydrogel for tissue growth according to the present disclosure as fabricated as described above includes a hydrogel body; pores formed within the hydrogel body; and a cell group positioned adjacent to the pores.

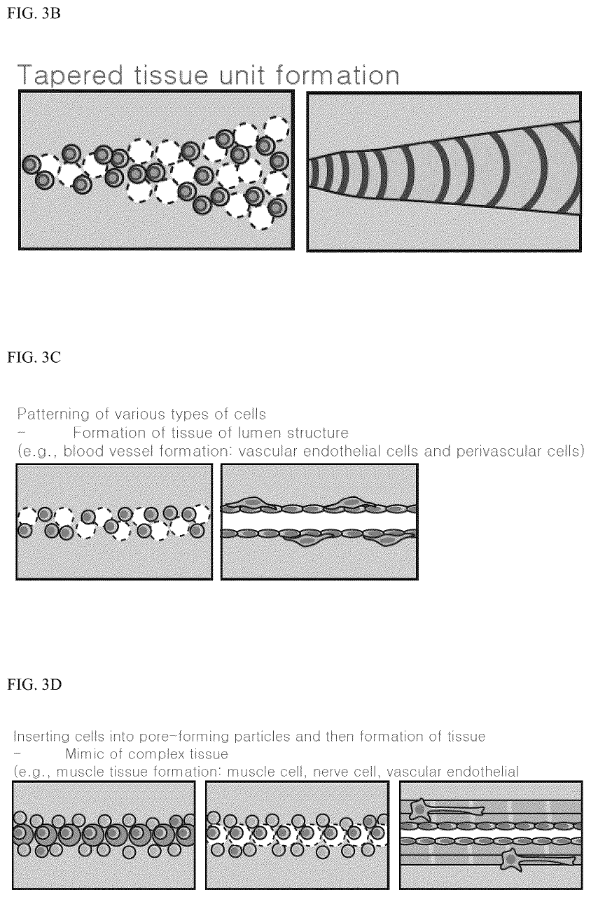

FIG. 3A to FIG. 3F show various types of patterned tissue units.
(4) Principle of controlling blood vessel thickness

Referring to FIG. 3A, the thickness of the tissue unit may be controlled by varying the concentration of the pore-forming particles or the cells.
(5) Fabricating various tissue shapes

Referring to FIG. 3B, the thickness of the tissue unit may be controlled by controlling the concentration of the pore-forming particles or the cells in a system that is configured to continuously extract the pore-forming particles and the cells while curing the gel.
(6) Patterning various types of cells

Referring to FIG. 3C and FIG. 3D, the pore-forming particles and two or more types of cells may be mixed with each other and the patterning may be performed thereon. For example, tissues with a lumen structure such as vascular endothelial cells and perivascular cells may be formed. The lumen-forming cells may be input into the pore-forming particles, and other types of cells may be mixed therewith, followed by the patterning to copy the tissue with a complex structure such as muscle cells, vascular endothelial cells, and nerve cells.
(7) Scaffold fabrication

Referring to FIG. 3E, a porous scaffold may be fabricated by patterning only the pore-forming particles without the cells. The scaffold may be used for transplantation, cell culture, etc.
(8) Rapid tissue formation induction

The growth factors may be added to the pore-forming particles, followed by the patterning, thereby fabricating a highly functional transplantation scaffold or forming a rapidly growing artificial tissue.

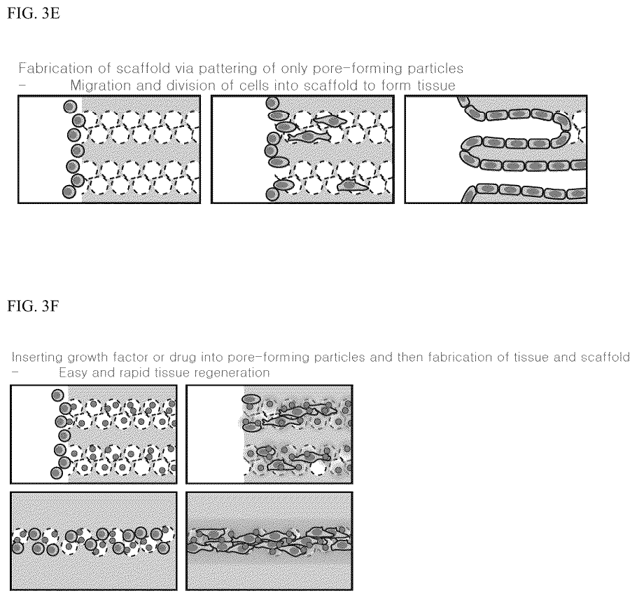

FIG. 4 is a schematic diagram of the artificial tissue fabricating device of the present disclosure.

Referring to FIG. 4, the artificial tissue fabricating device using acoustic patterning of pore-forming particles according to the present disclosure may include a chamber capable of receiving therein hydrogel, cells, and pore-forming particles; and an acoustic patterning unit capable of applying an acoustic wave to the chamber.

In one embodiment of the present disclosure, the acoustic patterning unit may include a piezoelectric element substrate, and an IDT (Interdigital transducer) electrode disposed on the piezoelectric element substrate.

In one embodiment of the present disclosure, the acoustic patterning unit may be configured such that an alternating current may be applied to the IDT electrode to generate the acoustic wave.

In one embodiment of the present disclosure, the acoustic wave may be a surface acoustic wave oscillating from the piezoelectric element substrate.

In one embodiment of the present disclosure, the acoustic wave may be applied from the acoustic patterning unit to the chamber such that the cells and the pore-forming particles may be patterned in one direction within the hydrogel.

In one embodiment of the present disclosure, the acoustic patterning unit may be configured to apply a standing wave to the chamber.

In one embodiment of the present disclosure, the artificial tissue fabricating device may further comprise an acoustic coupling medium further disposed between the acoustic patterning unit and the chamber.

Hereinafter, the artificial tissue fabricating device using acoustic patterning of pore-forming particles according to the present disclosure is described in detail.

Referring to FIG. 4, the artificial tissue fabricating device is composed of a piezoelectric element substrate, an IDT (interdigital transducer) electrode deposited on the piezoelectric element substrate, a PDMS (polydimethylsiloxane) microchannel (chip), a coupling solution, a coupling spacer, the hydrogel, and the pore-forming particles. The substrate and the IDT electrode generate and apply an acoustic wave corresponding to the 14 MHz or specific frequency which may transmit through the fluid. The frequency may be in a range of 1 to 100 MHz based on the sizes of the cells and the pore-forming particles. Surface acoustic waves of the same frequency respectively generated from both opposing sides are directed to the center area to generate the standing wave in the central chip area. The standing surface acoustic wave is transmitted to the hydrogel solution through the coupling medium (solution+spacer) and upper and lower coverslips of the chip. This process generates a standing pressure field in the solution. The PDMS microchannel (chip) is composed of a PDMS film and a coverslip. The PDMS film is cut into a frame shape to fit the size of the rectangular coverslip and then the frame is attached thereto. At this time, a cutting plotter or a punch is used. The upper end coverslip is attached onto the film. In this regard, the upper coverslip should be large enough to be attached to the film and be constructed to allow the solution to enter both opposing sides of the frame.

The artificial tissue fabricating device according to the present disclosure is configured such that the alternating current is applied to the electrode formed on the piezoelectric substrate to generate the surface acoustic wave. The generated surface acoustic waves are transmitted to the structure, the cells, and the solution through the acoustic coupling structure. The particles (beads) and the cells in the hydrogel are patterned at a regular spacing in the X and Z directions.

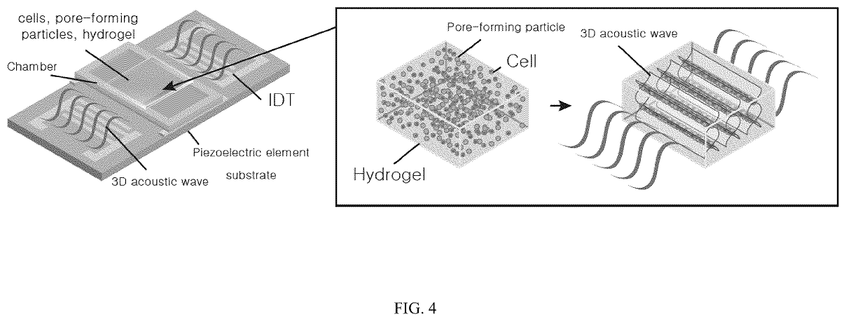

FIG. 5 is an illustration of the artificial vascular tissue regeneration.

Referring to FIG. 5, the cells move into the pores created by melting the beads patterned after the patterning and then divide. After the cells bind to each other, they grow into the tissue. Thus, a tissue structure in which the cells are densely arranged in a large volume is formed. (A) the pore-forming particles and the cells are patterned in one direction or unidirectionally in the hydrogel. (B) the pore-forming particles are dissolved and removed to form the pores. (C) the cells move toward the pores and grow. (D) the tissue is formed via the cell proliferation and adhesion. In particular, the pores are formed quickly in the pore-structured tissue.

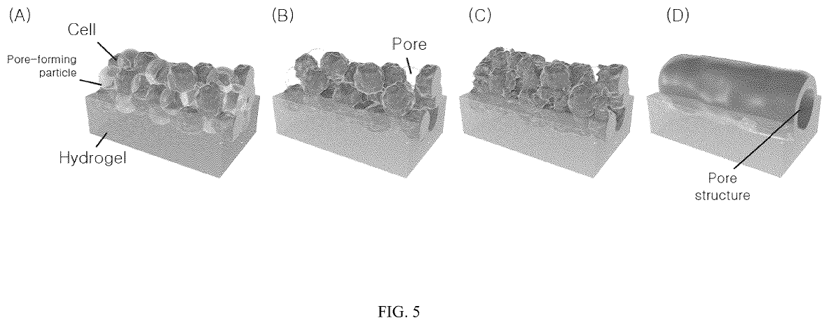

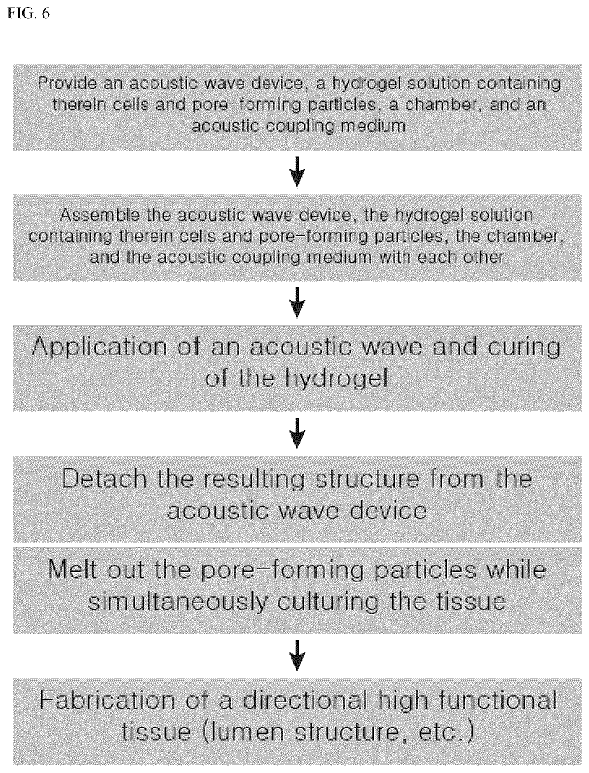

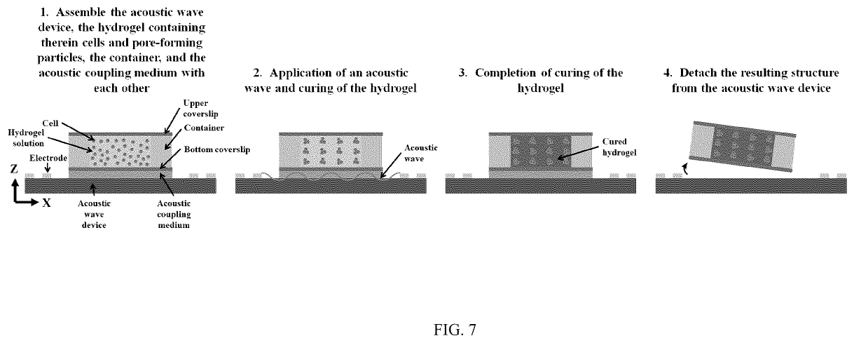

FIG. 6 is a flow chart of an artificial tissue fabricating process. FIG. 7 is a schematic diagram of an operation of the device during the artificial tissue fabricating process. FIG. 8 is a schematic diagram of structure decomposition and tissue extraction after tissue fabrication.

Referring to FIG. 6 to FIG. 8, first, a chamber including a base plate, a container, and a cover, an acoustic wave device, a hydrogel solution containing therein the cells and the pore-forming particles, and an acoustic coupling medium are prepared.

Then, the acoustic coupling medium is injected into a space defined on top of the acoustic wave device and under the chamber. The hydrogel solution containing the cells and the pore-forming particles is injected into the chamber. The acoustic wave device, the chamber, the hydrogel solution containing the cells and the pore-forming particles, and the acoustic coupling medium are assembled with each other.

Next, the acoustic wave application condition is set, and then, the acoustic wave is applied to the hydrogel solution containing the cells and the pore-forming particles through the acoustic coupling medium. Then, the hydrogel solution containing the cells and the pore-forming particles is cured. Thus, the artificial tissue fabrication time may be reduced by more than half due to the orientation of the cells toward the pores in which the cells penetrate a portion of the gel at a point where the gel is relatively easily deformable and move and grow.

Next, after the hydrogel solution containing the cells and the pore-forming particles has been cured, the resulting structure is detached from the acoustic wave device, and the tissue is cultured simultaneously while the pore-forming particles are eluted. In this regard, the pore-forming particles may be eluted at any time before or after the structure is detached therefrom.

Finally, the directional high-performance tissue is completed and extracted.

The structure including the fabricated tissue may be disassembled as shown in FIG. 8. First, the structure is detached from the acoustic wave device, and then the cover is removed therefrom, the container is removed therefrom, and the tissue is extracted therefrom. This order may be changed.

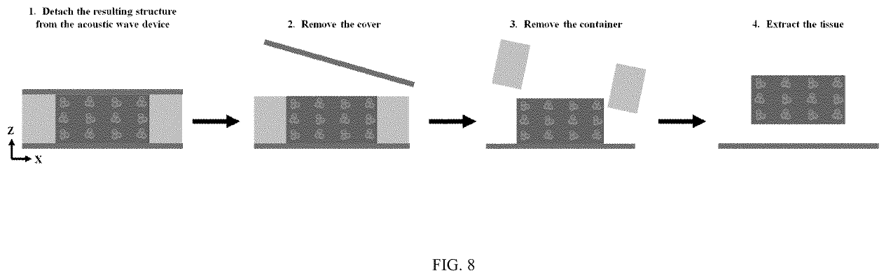

FIG. 9 is an image of the artificial vascular tissue fabricated according to the present disclosure, which is observed using a confocal microscope after staining actin as the cell structure with phalloidin.

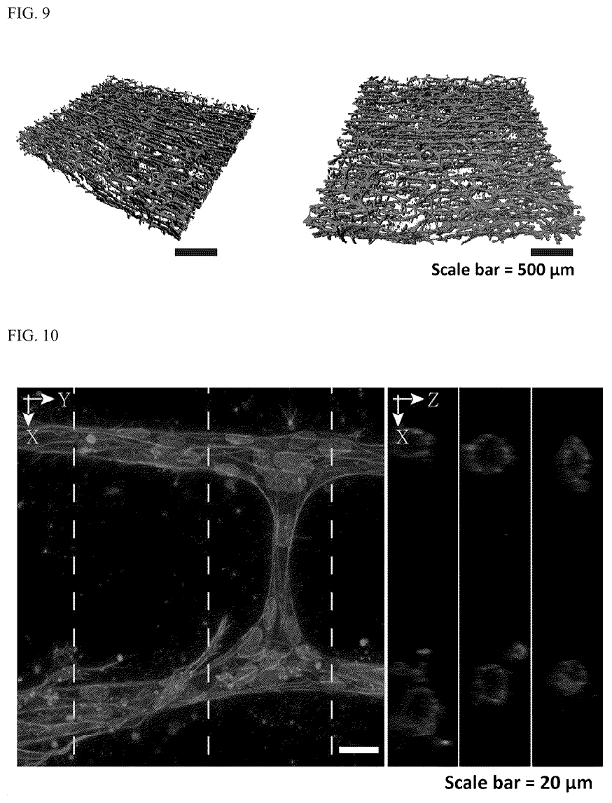

FIG. 10 is an image of the aligned microvascular structure after culturing the artificial vascular tissue fabricated according to the present disclosure for 2 days.

Referring to FIG. 10, the aligned microvascular structure may be observed after culturing the fabricated artificial vascular tissue for 2 days. The cross-section of each microvascular is enlarged to identify that the actin as the cell structure is stained in a shape of a hollow pipe, and the micropore structure as the unique characteristic of the blood vessels is clearly formed. In this regard, the pore structure is approximately 20 μm in diameter. The pore structure may be adjusted to a size of 10 to 100 μm depending on the size and the concentration of the pore-forming particles. Therefore, the pore-forming particle patterning scheme may allow the high-performance tissue to rapidly grow within 2 days.

1. A method for fabricating a hydrogel structure in which micro-sized pores are formed so as to be arranged in a direction of tissue growth, the method comprising:
a first step of mixing liquid hydrogel with cells and pore-forming particles;
a second step of applying an acoustic wave in one or more directions to the liquid hydrogel with which the cells and the pore-forming particles have been mixed, thereby aligning the cells and the pore-forming particles with each other;
a third step of curing the liquid hydrogel in which the cells and the pore-forming particles are aligned with each other, thereby forming a hydrogel structure; and
a fourth step of removing the pore-forming particles from the hydrogel structure to form pores inside the hydrogel structure.

2. The method for fabricating the hydrogel structure in which the micro-sized pores are formed so as to be arranged in the direction of tissue growth of claim 1, wherein the second step includes applying an acoustic wave to the liquid hydrogel to pattern the cells and the pore-forming particles.

3. The method for fabricating the hydrogel structure in which the micro-sized pores are formed so as to be arranged in the direction of tissue growth of claim 2, wherein the second step includes applying the acoustic wave to the liquid hydrogel to pattern the cells and the pore-forming particles in one direction.

4. The method for fabricating the hydrogel structure in which the micro-sized pores are formed so as to be arranged in the direction of tissue growth of claim 3, wherein the acoustic wave is a standing wave.

5. The method for fabricating the hydrogel structure in which the micro-sized pores are formed so as to be arranged in the direction of tissue growth of claim 1, wherein the hydrogel is cured before removing the pore-forming particles in the fourth step.

6. The method for fabricating the hydrogel structure in which the micro-sized pores are formed so as to be arranged in the direction of tissue growth of claim 5, wherein in the fourth step, the pore-forming particles are dissolved and removed using at least one of a solvent, heat, or light.

7. The method for fabricating the hydrogel structure in which the micro-sized pores are formed so as to be arranged in the direction of tissue growth of claim 6, wherein in the fourth step, the pore-forming particles are dissolved and removed using the heat.

8. The method for fabricating the hydrogel structure in which the micro-sized pores are formed so as to be arranged in the direction of tissue growth of claim 1, wherein the method further comprises a fifth step of growing cells into the pores obtained by removing the pore-forming particles, thereby forming a tissue.

9. A porous hydrogel for tissue growth, wherein the porous hydrogel is fabricated using the method for fabricating the porous hydrogel according to claim 1,
wherein the porous hydrogel for tissue growth comprises:
a hydrogel body;
pores formed within the hydrogel body; and
a cell group positioned adjacent to the pores.

10. An artificial tissue fabricating device using acoustic patterning of pore-forming particles, the artificial tissue fabricating device comprising:
a chamber capable of receiving therein hydrogel, cells, and pore-forming particles; and
an acoustic patterning unit capable of applying an acoustic wave to the chamber.

11. The artificial tissue fabricating device using acoustic patterning of pore-forming particles of claim 10, wherein the acoustic patterning unit includes a piezoelectric element substrate, and an IDT (Interdigital transducer) electrode disposed on the piezoelectric element substrate.

12. The artificial tissue fabricating device using acoustic patterning of pore-forming particles of claim 11, wherein the acoustic patterning unit is configured such that an alternating current is applied to the IDT electrode to generate the acoustic wave.

13. The artificial tissue fabricating device using acoustic patterning of pore-forming particles of claim 12, wherein the acoustic wave is a surface acoustic wave oscillating from the piezoelectric element substrate.

14. The artificial tissue fabricating device using acoustic patterning of pore-forming particles of claim 10, wherein the acoustic wave is applied from the acoustic patterning unit to the chamber such that the cells and the pore-forming particles are patterned in one direction within the hydrogel.

15. The artificial tissue fabricating device using acoustic patterning of pore-forming particles of claim 13, wherein the acoustic patterning unit is configured to apply a standing wave to the chamber.

16. The artificial tissue fabricating device using acoustic patterning of pore-forming particles of claim 10, wherein the artificial tissue fabricating device further comprises an acoustic coupling medium further disposed between the acoustic patterning unit and the chamber.
